(19) United States
(12) Patent Application Publication
(10) Pub. No.: US 2025/0378963 A1
(43) Pub. Date: Dec. 11, 2025

(54) System and Method for Personalized Health Optimization Using Causal Inference and a Dynamic Knowledge Graph

(71) Applicant: Lin Kiat Darren Soo, Markham (CA)

(72) Inventor: Lin Kiat Darren Soo, Markham (CA)

(21) Appl. No.: 19/218,402

(22) Filed: May 26, 2025

Related U.S. Application Data
(60) Provisional application No. 63/657,243, filed on Jun. 7, 2024.

(51) Int. Cl. G16H 50/30; G06N 5/022; G16H 40/67

(52) U.S. Cl. CPC G16H 50/30; G06N 5/022; G16H 40/67

(57) ABSTRACT

A computer-implemented system for personalized health optimization constructs a confidence-weighted personal health knowledge graph (PHKG) from heterogeneous data, including wearable sensors, medical devices, lab results, medication logs, and conversational inputs. A multi-stage causal-inference stack identifies modifiable drivers of outcomes using layered methods (e.g., MI, GAM, Neural Granger, DAG-GNN), and simulates candidate interventions. A recommendation engine ranks lifestyle or pharmacologic actions using a benefit-to-friction score, selecting a personalized intervention aligned with user readiness and clinical safety constraints. Interventions may include a minimum effective dose (MED), optimal level, adaptive low-dose, or behavioral challenge. Optional modules include reinforcement learning for timing adaptation and privacy-preserving on-device inference. The system operates across domains including metabolic, cardiovascular, renal, sleep, stress, and medication response, enabling cross-condition synergy evaluation. The architecture is modular, supports runtime plug-in targets, and adapts in real time with or without continuous clinical oversight, depending on deployment.

### CROSS-REFERENCE TO RELATED APPLICATIONS

This application claims priority to U.S. Provisional Patent Application No. 63/657,243, filed on Jun. 7, 2024, the entire contents of which are hereby incorporated by reference.

### FIELD OF THE INVENTION

The present invention relates to computer-implemented systems for personalized health management. More particularly, the invention concerns systems and methods that (i) ingest heterogeneous physiological, behavioral, environmental, and pharmacological data; (ii) represent such data in a hierarchical, time-stamped knowledge graph; (iii) apply multi-stage causal-inference techniques to discover and validate modifiable drivers across any health domain, and (iv) generate individualized, safety-constrained interventions whose dosage and timing are continuously adapted to the user's capacity and response. The system is agnostic to the specific condition(s) or biomarker(s) supplied, e.g., metabolic, cardiovascular, renal, sleep, stress, dermatological, or medication-response metrics, and operates whenever compatible markers and targets are provided.

### BACKGROUND OF THE INVENTION

Conventional digital-health solutions rely on static rules, population averages, or simple correlations. Such approaches exhibit several well-documented shortcomings:
Single-domain focus. Most applications optimize one metric or condition (for example, steps, glucose, or sleep) in isolation, ignoring clinically recognized comorbidities, e.g., the frequent co-occurrence of type 2 diabetes, hypertension, and obstructive sleep apnoea.

Delayed or generic medication titration. Pharmacological doses are typically adjusted only during infrequent clinical visits, using heuristics that overlook individual variability, lifestyle context, and cross-system side effects.

Fragmented data. Wearable streams, consumer medical-device readings, laboratory results, and patient-reported information remain siloed, precluding integrated causal reasoning.

Correlation bias. Without explicit causal modeling, existing systems cannot distinguish drivers from by-products, leading to ineffective or even contradictory recommendations.

Static interventions and poor adherence. Recommendations seldom account for user readiness, friction, or dose-response heterogeneity, and therefore fail to sustain behavioral change.

There exists a need for a unified system that: (i) incorporates any combination of biomarkers supplied by consumer devices (e.g., continuous-glucose monitors, photoplethysmography-based wearables, home blood-pressure cuffs) and clinical sources (e.g., laboratory haematology or renal panels); (ii) reasons causally across those data to reveal primary drivers, synergistic chains, amplifiers, and time-lagged effects spanning multiple conditions; (iii) quantifies user readiness and friction to recommend the minimum viable intervention (such as a minimum effective dose or lowest sufficient effort); and (iv) adapts dosage, including medication timing or amount, in near real time as new evidence accumulates. No known system provides this combination of multi-condition causal discovery, plug-in biomarker flexibility, and personalized dose optimization.

### Glossary

The following definitions apply throughout this specification.

“Friction” means a dimensionless score in [0,1] computed from adherence history and sentiment analysis, as described in
A=adherence history (normalized)
E=sentiment or engagement score

Formula:

$$Friction = 1 - ( w 1 * A + w 2 * E )$$

where w1 and w2 are tunable weights.

“Minimum Viable Intervention (MVI)” means the lowest-effort action whose predicted benefit-to-friction ratio exceeds a predefined threshold, as described in [0075].

“Minimum Effective Dose (MED)” is the lowest-effort intervention, in intensity or duration, whose expected benefit-to-friction ratio exceeds a system-defined threshold, as described in [0075].

Formula: An intervention i qualifies as MED if:

$$Benefit\_i / Friction\_i >= T\_MED where Benefit = Delta\_Y$$

“PHKG node confidence” means the Bayesian posterior probability that a node reflects a true observation, updated incrementally via evidence accumulation as described in [0077].

$$C = P ( node is valid \vert evidence )$$

Updated per: C_t=alpha*C_(t−1)+(1−alpha)*E_t, where E_t is new evidence confidence and alpha is between 0.7 and 0.9.

“Behavioral Readiness Score” means a scalar value computed from adherence, sentiment, and intent (R=f (adherence, sentiment, intent)) to reflect the user's capacity to adopt an intervention, per and as calculated in [0076].

$$R = ( A\_decay * 0.5 ) + ( S * 0.3 ) + ( I * 0.2 )$$

Where:
A_decay=7-day exponential adherence average
S=sentiment from NLP classifier (range −1 to +1, rescaled to [0,1])
I=self-reported intent [0-1]

“Causal edge weight” refers to The Bayesian posterior P (edge causal|evidence), updated via P_t=P_(t−1)*likelihood_ratio, where evidence includes Granger, CAM, DAG-GNN, and CF support. See [0078].

“Causal role” means the functional annotation assigned to each PHKG node to indicate its position in a causal graph. Causal role is selected from the group consisting of driver, amplifier, mediator, latent, and outcome, including but not limited to these roles.

“Synthetic-observation” refers to a simulated data point generated from user context and public datasets, and down-weighted relative to real data as described in [0079].

“Coefficient of Variation (CV)” is a normalized measure of variability:

$$CV = sigma / mu ,$$

Where sigma=standard deviation, mu=mean.

For sampling-uniformity thresholds, the CV may be set to a default value (e.g., 25%) in one embodiment, but can range from approximately 10% to 40% and is configurable per metric.

“Plug-in Schema” is a JSON-based runtime declaration identifying the target domain and metric for intervention optimization. Example:

```
{
  “domain”: “sleep”,
  “target_metric”: “REM_percent_min”,
  “goal”: 22.0
}
```

### SUMMARY OF THE INVENTION

In accordance with the foregoing need, the invention provides a modular, trade-name-agnostic health-optimization system comprising:

### Multi-Modal Data-Acquisition Layer

Receives time-stamped inputs from any mixture of (a) consumer wearables (heart-rate, heart-rate variability, respiration rate, sleep stages, step count, electrocardiogram), (b) consumer medical devices (continuous-glucose monitors, automated blood-pressure monitors, pulse oximeters), (c) clinical laboratory or electronic-health-record data (e.g., HbA1c, creatinine, lipid panel), (d) medication logs or pharmacy feeds (drug name, class, dose, administration schedule), and (e) user-provided or conversational inputs.

### Personal Health Knowledge Graph (PHKG)

Constructs, for each user, a directed, hierarchical graph whose nodes store metric values, timestamps, source type (direct, inferred, synthetic), and causal role (driver, amplifier, mediator, latent). Duplicate nodes are prevented; attributes are updated incrementally, ensuring a single canonical representation per metric.

### Post-PHKG Temporal-Inference Layer

Derives short- and long-range trends, spikes, lags, and event candidates (e.g., possible caffeine ingestion, medication-adherence event) not produced upstream, and appends them as low-confidence nodes for downstream evaluation.

### Causal-Targeted Intervention Module

Filters and prioritizes only those actions whose causal impact on key outcomes exceeds a tunable threshold, ensuring recommendations are both necessary and sufficient to shift root drivers rather than side effects.

### Personalization Engine

A Personalization Engine optionally models user readiness, intent, and adherence to dynamically select intervention difficulty or intensity. Optionally, a Behavioral Readiness Score

R=f(adherence,sentiment,intent)

may optionally guide configurable stages (e.g., MED to Optimal) depending on readiness modeling. Alternatively, context-sensitive tailoring strategies may be used without defined stages.

In some embodiments, the user's objective may be expressed as Reduction (improvement) or Maintenance (stability). The Personalization Engine can adapt intervention intensity or timing accordingly, either by escalating effort to drive improvement or by issuing occasional “booster” nudges to sustain stability.

### Multi-Stage Causal Inference Stack

The Multi-Stage Causal Inference Stack consists of:
(a) A priori modeling via generalized additive models (GAMs) to obtain interpretable dose-response curves;
(b) Statistical discovery using mutual information, Neural Granger causality, and additive-noise or CAM analyses;
(c) Structural confirmation with DAG-GNN learning and counterfactual simulation to quantify direct, indirect, and synergistic effects, including medication dose-response, across multiple conditions.

### Recommendation Engine

Ranks goal-aligned lifestyle or pharmacological interventions, optionally adapted to user-defined goals such as Reduction or Maintenance, by a friction-weighted benefit score, selecting the minimum viable intervention that satisfies safety limits and predicted efficacy. For maintenance-oriented objectives, interventions favor low-friction micro-actions; for reduction-oriented objectives, escalations toward higher-intensity actions are permitted if needed.

### Optional Reinforcement-Learning Module

In some embodiments, the system optionally further comprises a reinforcement-learning module that continuously tunes intervention timing, intensity, and fallback micro-challenges using objectively verified reward signals, and feeds resulting adherence/efficacy data back into PHKG edge confidences.

### Plug-In Use-Case Architecture

Condition-specific or medication-specific targets (e.g., HbA1c, systolic blood pressure, glomerular-filtration rate, minimum REM-sleep percentage, semaglutide dose) are declared at runtime. The core system processes these targets identically, enabling simultaneous optimization across diabetes, hypertension, sleep, stress, chronic-kidney disease, dermatological appearance, or medication efficacy without alteration to the underlying engine.

In this example, the user's goal for post_prandial glucose is set to <140 mg/dL; note that this is a user-defined target for the recommendation engine's optimization module and is not a safety constraint. Safety checks such as those defined in the Safety-Constraint Module at paragraphs [0062] to [0063], post-meal glucose walk threshold (>180 mg/dL) remain enforced independently of user goals.

### Standards-Based Interoperability

The system optionally supports integration with third-party systems via standards-based interfaces, including HL7 FHIR (Fast Healthcare Interoperability Resources), to allow secure communication with clinical electronic health records, employer-sponsored wellness systems, or insurer dashboards. This facilitates bidirectional flow of lab data, medication logs, and user progress across institutional systems.

In one aspect, the invention therefore furnishes a computer-implemented system that, upon receiving heterogeneous health inputs, constructs a confidence-weighted PHKG, executes a layered causal-inference pipeline, and outputs personalized, phase-appropriate interventions that may include adjusted medication dosing. In another aspect, the invention provides a method for using such a system to improve multiple co-existing health conditions concurrently, while accounting for user-specific readiness and friction. Optional embodiments include on-device threshold detection, federated inference, and privacy-preserving deployments.

Importantly, the engine is non-linear and cross-condition aware: interventions proposed for one domain (e.g. glucose control) are simulated for downstream impacts on other domains (e.g. blood pressure), and only those that enhance or neutrally affect co-morbid outcomes are advanced-addressing cross-domain causality in a single pass.

### BRIEF DESCRIPTION OF THE DRAWINGS

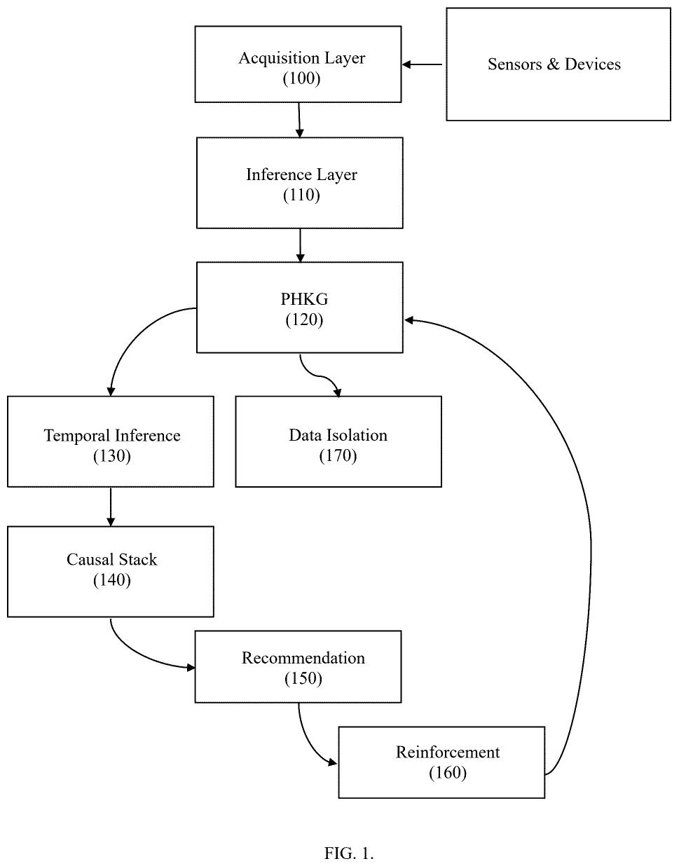

FIG. 1 illustrates a high-level architecture of the system, including:
the multi-modal data-acquisition (100) layer for ingesting time-stamped physiological, behavioral, and pharmacological inputs;
the Personal Health Knowledge Graph (PHKG) (120) engine for constructing and updating directed, confidence-weighted nodes and edges;
the multi-stage causal-inference stack (140) for GAM modeling, statistical discovery (MI, Neural Granger, CAM), and structural confirmation (DAG-GNN, counterfactual simulation);
the recommendation engine (150) for computing benefit-to-friction scores and selecting safety-constrained interventions.

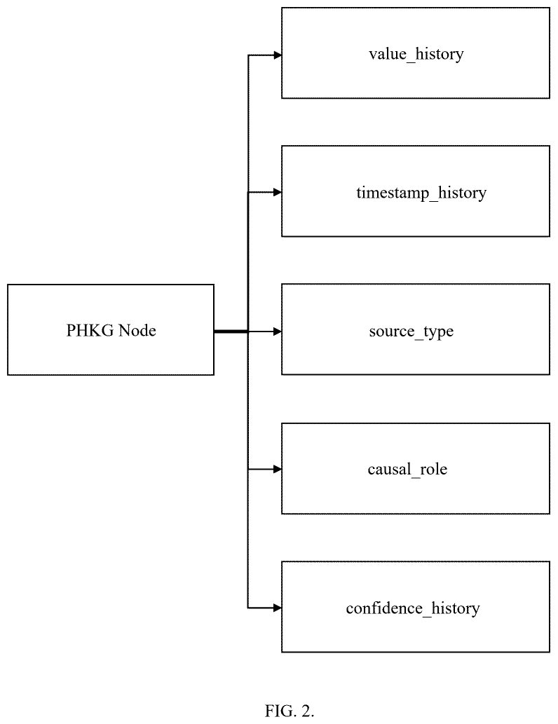

FIG. 2 depicts the schema for a PHKG node, showing:
the <value_history> array capturing historical metric values;
the <timestamp_history> array for corresponding timestamps;
the <source_type> label (direct, inferred, synthetic);
the <causal_role> attribute (driver, amplifier, mediator, latent, outcome, etc.);
the <confidence_history> array storing Bayesian-updated belief scores.

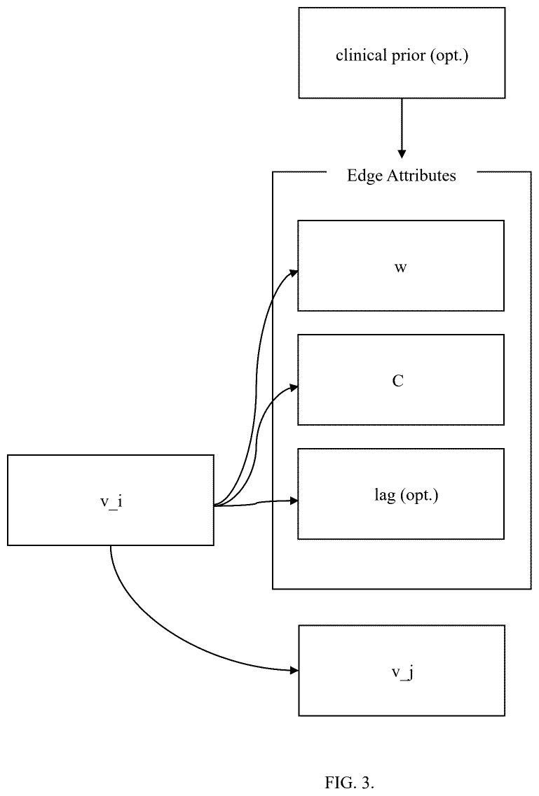

FIG. 3 shows the causal edge structure between two nodes, including:
the directed edge from node v_i to v_j;
the <causal_weight> w quantifying effect strength;
the C representing edge veracity;
optional <lag_distribution> metadata;
pre-seeded clinical priors illustrating known biomedical relationships.

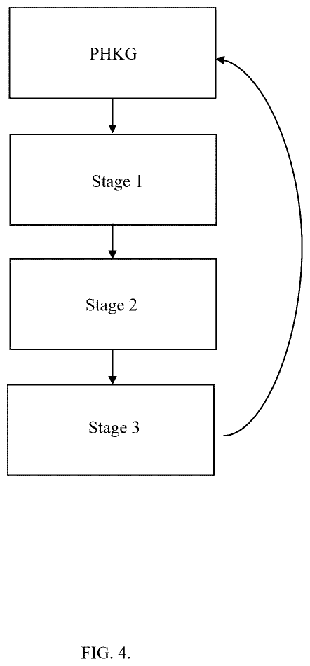

FIG. 4 outlines the multi-stage causal-inference pipeline, comprising three stages:
Stage I: GAM modeling to fit interpretable dose-response curves;
Stage II: statistical discovery via mutual information screening, Neural Granger causality tests, and additive-noise (CAM) analyses;
Stage III: structural confirmation using a DAG-GNN to learn minimal causal edges, followed by counterfactual simulation to estimate intervention effects and update edge confidences.

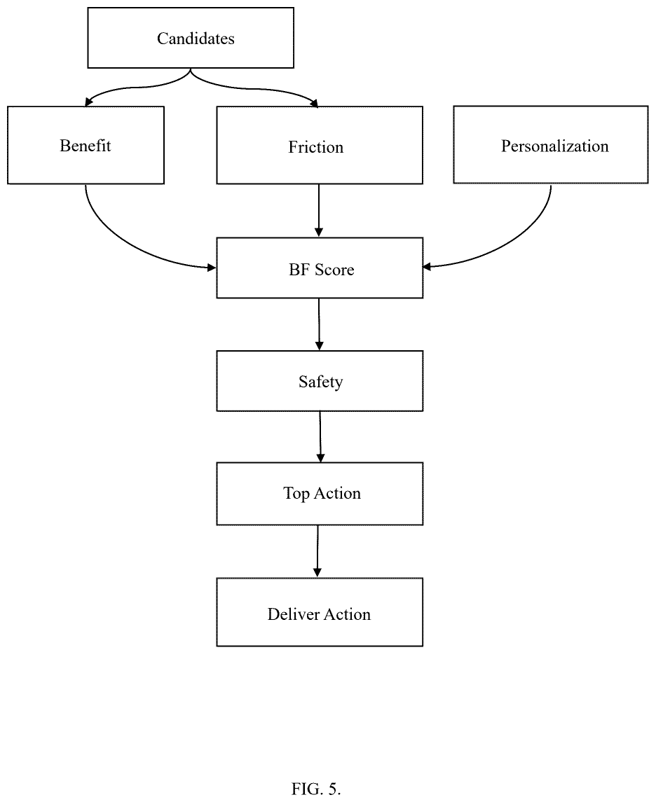

FIG. 5 diagrams the recommendation engine workflow, showing:
candidate interventions as input;
computation of predicted causal benefit (Delta_Y/sigma) and user-specific friction (derived from adherence history and sentiment analysis);
ranking of Benefit/Friction scores;
enforcement of clinical safety constraints (e.g., max HRR, blood-glucose limits);
selection and output of the minimum viable intervention (MED, optimal effort, adaptive low-dose, or hybrid bundle).

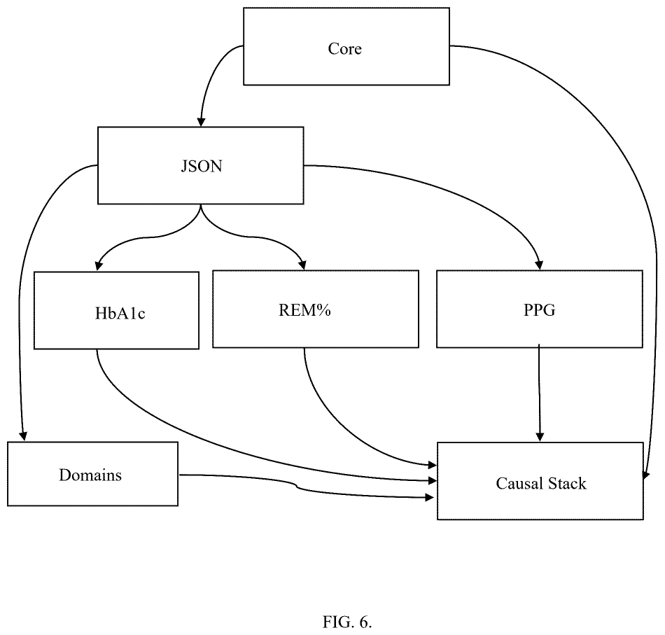

FIG. 6 presents an example of the plug-in architecture for multi-condition optimization, illustrating:
the core system and causal stack shared across domains;
runtime JSON schema declarations for different health targets (e.g., HbA1c goal for diabetes, REM_percent_min for sleep, post_prandial_glucose for glycemic control);
parallel processing of multiple domain-specific objectives without code modifications.

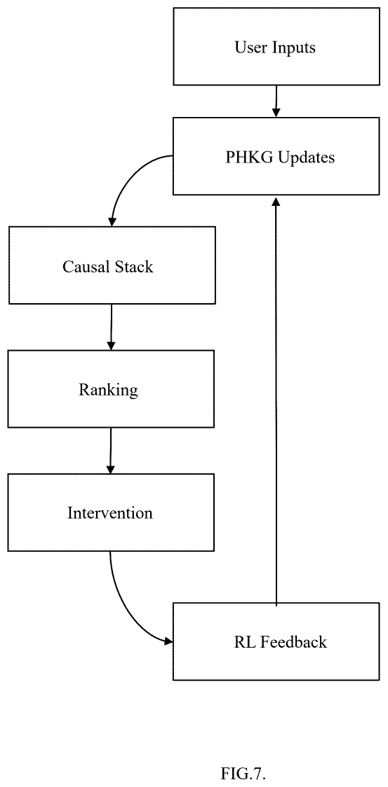

FIG. 7 illustrates an end-to-end use-case flow, depicting:
input streams from wearables, lab data, medication logs, and user self-reports;
PHKG node and edge updates in response to new evidence;
identification of causal drivers and chains via the multi-stage inference stack;
ranking and selection of personalized interventions by the recommendation engine;
reinforcement-learning adaptation based on adherence feedback and outcome verification.

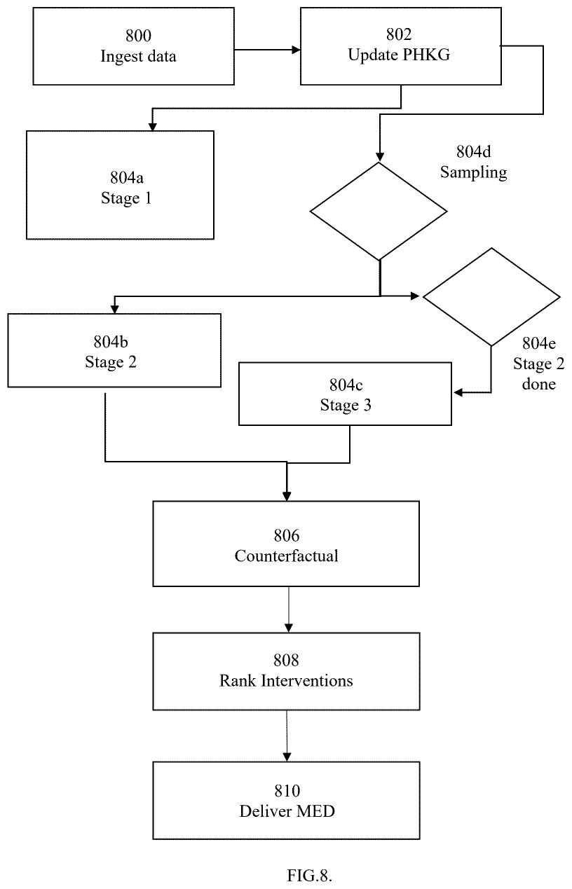

FIG. 8 depicts the intervention execution flowchart, showing sequential method steps:
800: ingest multi-modal data from sensors, devices, and conversational inputs;
802: build or update the PHKG in memory;
804: execute the causal-inference stack (GAM to MI to CAM to DAG-GNN);
806: run counterfactual simulation and Bayesian confidence update;
808: rank candidate interventions by Benefit/Friction scores;
810: deliver the selected intervention (MED, Optimal, or tailored action, etc) and log feedback for audit and future reinforcement learning.

FIG. 8 further includes the following decision diamonds:
804a gating entry to Stage 1 (GAM modeling),
804b gating entry to Stage 2 (statistical discovery),
804c gating entry to Stage 3 (structural confirmation),
804d, verifying that both sample-count and CV thresholds are met before entering Stage 2 or Stage 3.
804e verifying that Stage 2 has executed at least once before entering Stage 3.

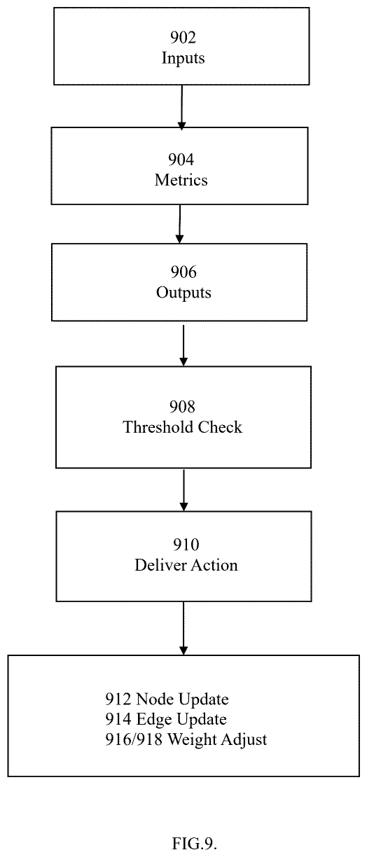

FIG. 9 illustrates the computational data-flow for intervention selection, showing how PHKG node inputs are used for key metric calculations (friction, benefit, readiness), followed by threshold comparison, intervention delivery and PHKG confidence update cycles:
902: ingestion of physiological and behavioral data streams;
904: calculation of key metrics including adherence, sentiment, friction, and predicted benefit,
906: evaluation of the Benefit-to-Friction ratio and comparison to the Minimum Effect Dose (MED) threshold,
908: comparison of Benefit/Friction ratio to the system-defined MED threshold,
910: delivery of selected intervention and feedback logging,
and 912-918: updates to PHKG node and edge confidence using the Bayesian methods detailed in the specification.

### DETAILED DESCRIPTION OF THE INVENTION

The following description is provided to enable any person skilled in the art to make and use the invention. Numerous specific details are set forth to facilitate understanding; however, the invention may be practiced without these specific details. Where appropriate, like reference numerals refer to like elements.

### System Overview

FIG. 1 illustrates an exemplary architecture comprising: (i) a Multi-Modal Data-Acquisition Layer (100); (ii) a Wearable & Contextual Inference Layer (110); (iii) a Personal Health Knowledge Graph Engine (PHKG) (120); (iv) a Post-PHKG Temporal-Inference Module (130); (v) a Multi-Stage Causal-Inference Stack (140); (vi) a Recommendation Engine (150); and optionally (vii) a Reinforcement-Learning Module (160). A secure data architecture (170) isolates personally identifiable information (PII) from personal health information (PHI). Each layer is described below.

### Method Overview (FIG. 8)

Referring to FIG. 8, step 800 ingests wearable and clinical inputs. Step 802 constructs or updates the PHKG in memory. Step 804 invokes the multi-stage causal-inference stack (see [0057]-[0059]). Block 804a executes Stage 1 causal-inference pass (PHKG→GAM) on every PHKG update. At decision diamond 804d, the system checks whether the data meet the Stage 2 thresholds as defined in [0048]-[0050]; if “No”, control loops back to block 802 for the next PHKG update, and if “Yes”, control passes to block 804b to execute Stage 2 (MI→GAM→Neural Granger→CAM).

After block 804b completes, decision diamond 804e verifies that Stage 2 has run at least once and that the data also meet the Stage 3 thresholds as defined in [0048]-[0050]; if “Yes”, control advances to block 804c (Stage 3: DAG-GNN→deep counterfactual). Outputs of 804b and 804c both flow into block 806 for the Bayesian counterfactual evidence update, followed by blocks 808 and 810 for ranking and delivery of interventions.

### Minimum Data Requirements for Layered Causal Analysis

The system applies stage-specific data-sufficiency criteria to gate each layer of causal inference, ensuring statistical reliability at every level. Let Delta_i=T_(i+1)−T_i denote the sampling interval between consecutive timestamps for a given metric. The coefficient of variation (CV) of the set Delta is defined as CV=(sigma (Delta)/mu(Delta))*100% where sigma and mu are the standard deviation and mean of the sampling intervals, respectively. A lower CV indicates more uniform spacing of observations.

| Causal-Inference | | |
|---|---|---|
| Stage | Default Trigger | Rationale |
| Stage 1 | At least two (2) | A Generalized Additive Model |
| (GAM preview) | valid samples (no | can fit an initial dose response |
| | CV requirement). | curve with as few as two data |
| | | points. |
| Stage 2 (MI to | At least seven (7) | MI and Neural Granger require |
| GAM to Neural | valid samples within | multiple lagged pairs; seven |
| Granger to CAM) | a rolling seven-day | evenly spaced points satisfy this |
| | window and | minimum. Stage 2's GAM |
| | CV <= 25%. | refinement imposes no |
| | | additional lag requirements. |
| Stage 3 | At least thirty (30) | Thirty observations over two |
| (DAG-GNN | valid samples within | weeks provide sufficient data for |
| to Deep | a rolling fourteen- | graph-neural convergence and |
| Counterfactual) | day window and | stable causal estimates. |
| | CV <= 25%. | |

These thresholds are implemented as modifiable parameters (stage1_min_count, stage2_min_count, stage2_max_cv, stage3_min_count, stage3_max_cv) stored in non-transitory memory, with the defaults above. Updates to these parameters (for example, reducing the Stage 2 count to five samples for high-frequency streams) do not depart from the scope of the invention, as claim 8 anchors the invention to the presence of stored threshold parameters and their default values.

### Implementation Example (Python-Like Pseudo-Code)

The following pseudo-code shows one way to implement the stage-gated thresholds on a standard cloud server (e.g, 2 vCPU, 8 GB RAM) executing the above on 100 metrics completes at Stage 1 pass in approximately 30 milliseconds, Stage 2 in approximately 250 milliseconds and Stage 3 in approximately 1.5 seconds, demonstrating feasibility without specialized hardware or GPUs.

| | |
|---|---|
| import numpy as np | # illustrative dependency |

# defaults stored in non-transitory memory
stage1_min_count = 2

| | |
|---|---|
| stage2_min_count = 7 | # default; may be tuned. |
| stage2_max_cv = 0.25 | # default; may be tuned 0.10-0.40 |

stage3_min_count = 30

| | |
|---|---|
| stage3_max_cv = 0.25 | # default; may be tuned 0.10-0.40 |

```
def coeff_variation(ts):
  delta = np.diff(sorted(ts))
  return np.std(delta) / np.mean(delta)
# run_GAM, run_MI, etc. are module calls defined elsewhere
def causal_pipeline(metric):
  if metric.n >= stage1_min_count:
    run_GAM(metric)
  if (metric.n >= stage2_min_count
    and coeff_variation(metric.timestamps) <= stage2_max_cv):
    run_MI(metric)
    run_NeuralGranger(metric)
    run_CAM(metric)
  if (metric.n >= stage3_min_count
    and coeff_variation(metric.timestamps) <= stage3_max_cv):
    run_DAGGNN(metric)
    run_counterfactual(metric)
```

Empirical validation (Table 5-A) shows that enforcing the Stage 3 thresholds reduces false-positive causal edges below 5%, whereas relaxing either the count or CV gates raises false positive to approximately 15%. Therefore, the gating logic is essential for delivering reliable, personalized interventions.

### See Table 5-A:

TABLE 5-A
Empirical False-Positive Rates with and without Stage 3 Gating

| Threshold Configuration | False-Positive Rate (%) |
|---|---|
| No Stage 3 gating (any >= 5 samples) | 14.94% |
| Stage 3 gating (>=30 samples & CV <= | 4.83% |
| 25%) | |

### Multi-Modal Data-Acquisition Layer (100)

The acquisition layer receives time-stamped data selected from:
Wearable sensors—heart-rate, heart-rate variability, respiration, sleep stages, step count, electrocardiogram, oxygen saturation.
Consumer medical devices—continuous-glucose monitors, automated blood-pressure monitors, digital spirometers.
Clinical sources—laboratory panels (e.g., HbA1c, creatinine, lipid profile), pharmacy or electronic-health-record feeds.
Pharmacological logs—drug name, class, dose, administration route, schedule, refill adherence.
Contextual or conversational inputs—stress self-reports, dietary entries, free-text dialogue.
   Data are normalised to unified units and written to a transient buffer for upstream inference.

### Wearable & Contextual Inference Layer (110)

In one embodiment, proprietary heuristics convert raw signals into low-level events (e.g., heart-rate spike, sleep-fragment episode, possible caffeine intake). Each inferred event is emitted as a JSON object comprising: {metric_name, value, timestamp, source=“inferred”, confidence}. This layer may execute on-device or in the cloud; its internal thresholds are implementation-agnostic and need not be disclosed.

### Best-Mode Safe Harbor

The inventors' preferred wearable-inference implementation is proprietary and may involve rule-based heuristics or hybrid logic. However, any method that emits timestamped, confidence-weighted events from raw wearable signals shall suffice for practicing the invention.

### Personal Health Knowledge Graph Engine (120)

Node Schema: Each user graph G_u=(V, E) maintains a single canonical node v is in V for each metric or event. Every node stores an attribute vector:
[value_history[ ], timestamp_history[ ], source_type, role, confidence_history[ ]], where
   source_type is in {direct, inferred, synthetic}.
   role is selected from the group consisting of driver, amplifier, mediator, latent, and outcome, including but not limited to these roles.
   confidence_history records Bayesian-updated belief in the node's veracity.

Edge Schema: Directed edge e=(v_i, v_j, w, C) links nodes v_i→v_j with causal weight w and confidence C. Edges are updated incrementally; duplicates are prevented by hashing (v_i, v_j). Clinically grounded priors may be pre-seeded (e.g., “systolic blood-pressure to stroke risk”).

### Post-PHKG Temporal-Inference Module (130)

This module computes windowed statistics (min, max, mean, sigma), rolling slopes, changepoints, and lag correlations over the value_history[ ] arrays, emitting new trend nodes (e.g., “7-day HRV decline”) or event candidates (“possible medication non-adherence”) flagged with low initial confidence for downstream causal validation.

### Multi-Stage Causal-Inference Stack (140)

### Stage I—A Priori Modeling

Generalised additive models (GAMs) fit monotonic or spline dose-response curves f_k(x) between candidate drivers and outcomes, producing interpretable beta-coefficients.

### Stage II—Statistical Discovery

Mutual-information screening, Generative additive modeling (GAM), Neural Granger causality, and additive-noise (CAM) tests identify directionality and temporal precedence, pruning spurious links.

### Stage III—Structural Confirmation

A directed-acyclic-graph neural network (DAG-GNN) learns the minimal edge set consistent with Stages I-II. A deep counterfactual simulator, Sim(x), perturbs candidate driver nodes delta_x and estimates Delta_Y across multi-hop paths, returning {Delta_Y, sigma\hat{ }2, path_confidence}. This deep counterfactual is distinct from Bayesian counterfactual used for edge-confidence updates in claim 7. Causal-effect estimates are also evaluated across domains, such that if a candidate intervention improves one target (e.g., glucose) but worsens another (e.g., sleep or blood pressure), it is downgraded or suppressed unless explicitly allowed. The system prioritizes multi-domain utility by computing a joint optimization score.

### Default Hyperparameter Settings

The following illustrative parameters enable the causal stack for general-purpose health metrics:
Granger max lag (tau): 5.
MI kernel width (sigma): 1.2.
DAG-GNN hidden size: 64.
Learning rate (alpha): 1e-3.
Q-learning discount factor (gamma): 0.95.
Exploration epislon schedule: linear decay from 1.0 to 0.05 over 10 k iterations.
   These settings are non-limiting. Any equivalent values or architectures that produce valid causal graphs or policy updates are compatible with the invention.

### Medication Modeling (Plug-In Example)

### Medication Events are Represented as Nodes:

med_node=[drug_class, dose, route, schedule, timestamp, source_type=“direct”, role-“driver”]. Edges connect med_node to physiological or behavioral nodes (e.g., “semaglutide to post-prandial glucose,” “beta-blocker to HRV”). Lagged effects are modelled by storing lag_distribution metadata on each edge. Counterfactual simulation evaluates alternative doses or administration times to compute DeltaOutcome and recommend titration that satisfies minimum viable therapeutic dose (e.g., MED) while minimising amplifiers (e.g., sleep disturbance).

### Safety-Constraint Module

In one embodiment, the recommendation engine enforces the following clinically grounded limits before issuing any reinforcement learning (RL)-guided or causal-driven intervention.

The numerical thresholds and adjustment factors below are illustrative only; any clinically accepted limits or condition-specific modifications may be substituted in alternate embodiments without departing from the scope of the invention.
1. Max Heart-Rate (HR) Zone

$$Maximum HR ( MHR ) = ( 208 - 0.7 * age ) * HCF$$

where HCF (Heart Condition Factor) equals 1.00 for users without cardiovascular disease and 0.85 for users with a diagnosed cardiovascular condition.
This formulation is based on population norms as described by Tanaka et al., “Age-Predicted Maximal Heart Rate Revisited,” Journal of the American College of Cardiology, 2001; 37 (1):153-156.

$$Heart - Rate Reserve ( HRR ) = MHR - resting\_HR .$$

Target Intensity Zones:

$$Moderate : 40 \% - 59 \% of HRR + resting\_HR .$$

$$Vigorous : 60 \% - 85 \% of HRR + resting\_HR .$$

These exercise zones are consistent with the published clinical guidelines from the American College of Sports Medicine (ACSM), as stated in ACSM's Guidelines for Exercise Testing and Prescription, 11th ed., 2021.
Absolute upper limit: never exceed 85% of HRR
2. Workout Duration & Frequency
   Initial: 10 minutes/session at moderate intensity (40%-59% HRR), 3 sessions/week.
   Progression: increase by 5 minutes per week until 30 minutes/session.
   Long-Term Goal: up to 5 sessions/week aerobic+2 sessions/week strength training.
3. Post-Meal Glucose Walk
   If 2-hour post-prandial blood glucose (RBG)>180 mg/dL, the system recommends a 10 minute walk at an intensity <=40% of HRR.
   This threshold aligns with the American Diabetes Association's 2024 target for postprandial glycemic control in non-pregnant adults, as published in the Standards of Medical Care in Diabetes—2024, Diabetes Care, 47 (Suppl. 1): S1-S215.
4. Micro-Challenge Triggers
   HRV-Based Breathing: if inferred HRV< (baseline-15%), prompt 5 minutes paced breathing at 4-6 breaths/minute.
   Sleep Hygiene: if sleep fragmentation>2 awakenings/night, prompt “screen-off” 30 minutes before bedtime.

### Recommendation Engine (150)

For each intervention i the engine computes:

$$Benefit\_i = abs ( Delta\_Y \_i ) / sigma\_i ( predicted effect size / uncertainty ) ,$$

Friction_i is a value between 0 and 1, inclusive (derived from adherence history and sentiment), and ranks candidates by Benefit_i/Friction_i. The top-ranked action satisfying safety limits, as defined in [0062]-[0063], (e.g., max HR, BP thresholds) is selected as minimum viable intervention (e.g., Minimum Effective Dose (MED), adaptive low-dose, or lowest sufficient effort). Interventions may span lifestyle (sleep cutoff, step increment), medication adjustments (dose or timing shift), or hybrid bundles (e.g., 250-step walk plus 2-hour dose delay).

### Sparse-Data Fallback Logic

In accordance with claim 8 and the paragraphs to data-sufficiency gating, each causal-inference stage only executes when its stored sample-count and sampling-uniformity criteria are satisfied. If the active target metric (or any driver candidate) fails to meet the gating criteria for Stage II or Stage III, i.e., sample_count or CV thresholds stored in non-transitory memory, then the engine bypasses those stages and instead performs one or more of:
1. Rule-based heuristics; or
2. Single-variable GAM prediction
   until the corresponding sample-count and sampling-uniformity criteria are met. During this fallback, the recommendation engine continues delivering low-risk, rule-based interventions to preserve continuity of care.

### Reinforcement-Learning Module (160)

A Q-learning policy pi(s, a) receives a state vector (phase, adherence, sentiment, engagement history, edge-confidence drift) and selects intervention timing, intensity, or fallback micro-challenges.

Behavioral & emotional adaptation. If repeated non-compliance, low motivation, or negative sentiment (e.g., frustration detected via conversational analysis) is observed, the policy reduces friction by (i) lowering the MED dose, (ii) issuing an easier “bait” challenge (e.g., “walk 100 steps now”), or (iii) pausing suggestions until sentiment improves. Positive sentiment or sustained adherence may cause the policy to escalate intervention intensity, expand action types, or deliver more ambitious challenges, if appropriate to the user's profile or goal.

Reward function. Objective reward R=w1*Delta Outcome+w2*Adherence+w3*Engagement−w4*UserReportedBurden, where w1-w4 are tunable weights. Negative reward lowers intervention frequency or difficulty, while positive reward reinforces the current policy. Edge confidences in the PHKG are updated with every validated outcome, providing a closed feedback loop between causal inference and behavioral reinforcement.

### 5.11 Plug-In Architecture

Condition-specific targets are declared at runtime via a JSON schema:

```
{
  “domain”: “sleep”,
  “target_metric”: “REM_percent_min”,
  “goal”: 22.0
}
or
{
  “domain”: “medication”,
  “drug”: “semaglutide”,
  “target_metric”: “post_prandial_glucose”,
  “goal”: “less than 140 mg/dL”
}
```

The causal stack treats each declaration identically, permitting simultaneous optimization across any combination of diabetes, hypertension, sleep, chronic-kidney disease, stress, appearance, or medication efficacy without code modification.

### Illustrative End-To-End Example

1. Input Streams. Wearable HRV, home CGM glucose, lab HbA1c, semaglutide 5 mg weekly dose, self-reported stress.
2. PHKG Update. Nodes for HRV, glucose, HbA1c, semaglutide dose updated; edge “semaglutide→glucose” initial confidence 0.7.
3. Causal Analysis. Stack identifies: (i) insufficient REM sleep mediates semaglutide efficacy; (ii) 4-hour dose-time shift predicts-12 mg/dL post-prandial glucose (sigma=2).
4. Recommendation. Engine ranks:
   a. Shift dose from 22:00 to 18:00 (Benefit/Friction=3.2)
   b. Add 250 evening steps (2.5)
   c. Screen-off 30 min pre-bed (1.9) the minimum viable intervention (e.g., MED); fallback=250-step walk.
5. RL Adaptation. Adherence verified via pharmacy refill+accelerometer; RL increases confidence of dose-time edge to 0.85 and suppresses low-yield screen-off reminders.

### Key Metric Calculations and Evidentiary Description

This section consolidates the quantitative definitions, variable sources, and exemplary calculations for the core decision variables employed throughout the system. Unless stated otherwise, all symbols retain the meanings introduced in the Glossary. Where appropriate, illustrative values are provided to demonstrate enablement; these values are non-limiting and may be scaled or tuned without departing from the scope of the claims.

Friction Score. Friction is a dimensionless burden indicator in the closed interval [0, 1] computed as:

$$Friction = 1 - ( w 1 * A + w 2 * E ) .$$

Adherence history (A) is the proportion of completed, logged interventions in a rolling seven-day window (e.g., 0.0-1.0). Sentiment/engagement (E) is a natural-language sentiment score extracted from conversational feedback or button presses, mapped from [−1, +1] to [0, 1]. Default weights are w1=0.5, w2=0.2, but may be personalised by reinforcement learning.

Example. If A=0.70, E=0.60, Friction=1−(0.5*0.70+0.2*0.60)=1−0.47=0.53, indicating moderate user burden.

Predicted Benefit (Delta_Y) and Uncertainty (sigma). Delta_Y denotes the counterfactual change in a target metric predicted by the Stage-III simulator when an intervention vector is applied versus withheld. Sigma is the model-estimated standard deviation of that prediction. A benefit score is derived as:

$$Benefit = abs ( Delta\_Y ) / sigma$$

Example. Simulated evening-dose shift predicts Delta_Y=−12 mg/dL for post-prandial glucose with sigma=2 mg/dL, yielding Benefit=6.

Minimum Effective Dose (MED) and Minimum Viable Intervention (MVI). An intervention qualifies as MED when

$$Benefit / Friction >= T\_MED ,$$

where T_MED is a tunable threshold (default=1.5). The minimum-viable intervention is the lowest-effort action or bundle meeting the MED constraint while satisfying all safety limits as defined in [0062] through [0063]. Example. Using the Friction example from (0.29) and Benefit=6 (from [0074]): Benefit/Friction=20.7>>1.5, so the dose-shift intervention meets MED and, if lower-effort than alternatives, becomes the MVI.

Behavioral Readiness Score. Readiness is computed as

$$R = ( A\_decay * 0.5 ) + ( S\_sent * 0.3 ) + ( I\_intent * 0.2 ) ,$$

where A_decay is the 7-day exponentially weighted adherence average (lambda approximate 0.3), S_sent is current sentiment rescaled to [0, 1], and I_intent is self-reported intent (0-1). Example. A_decay=0.60, S_sent=0.90, I_intent=0.80→R=0.73, suggesting high readiness to escalate intensity.

PHKG Node Confidence Updating. Each node's posterior confidence is updated after new evidence arrives by

$$C\_t = alpha * C\_ ( t - 1 ) + ( 1 - alpha ) * E\_t ,$$

alpha is between 0.7 and 0.9.

E_t represents evidence quality: direct laboratory measurement (1.0), wearable sensor reading (0.7), inference (0.5), synthetic observation (0.3).

Example. With C0=0.60, alpha=0.80, and a wearable step count record E1=0.70: C1=0.80*0.60+0.20*0.70=0.64.

Causal Edge Confidence Updating. Directed edge confidence P_t is multiplicatively adjusted by a likelihood ratio (LR) derived from new causal evidence:

$$P\_t = P\_ ( t - 1 ) * LR , LR > 0.$$

LR values are heuristically set: Neural Granger pass (1.2), CAM confirmation (1.1), DAG-GNN persistence (1.3), counterfactual validation (1.4). If conflicting evidence arises, LR may be <1. Example. Edge “resting_heart_rate→post-prandial_glucose” has P0=0.50. After DAG-GNN learning (LR=1.3) and counterfactual support (LR=1.4): P=0.50*1.3*1.4=0.91.

Synthetic-Observation Weighting. Synthetic data are generated from user profile context and public cohorts to fill sparse windows. A synthetic weight W_synth<=0.30 is applied in all Bayesian updates. Once the real-data sample count for a metric exceeds the Stage-3 threshold (30 samples, [0048]-[0050]), W_synth decays toward zero by W_synth(new)=W_synth (prior)*exp (−k*(N_real−30)), k is approximately 0.1. Example. If W_synth=0.30 when N_real=30, then at N_real=40: W_synth is approximately equal to 0.30*e\hat{ }(−0.1*10) approximately equal to 0.11.

Integrated Example. Table 5-B illustrates a typical end-to-end cycle for a 45-year-old male user over 24 hours. Wearable sensors record adherence of 8/10 prescribed micro-walks (A=0.80) and favourable sentiment (+0.6→S_sent=0.80), yielding Friction=0.2. The causal stack predicts Delta_Y=−8 mg/dL with sigma=2 mg/dL (Benefit=4). Benefit/Friction=12.9 surpasses T_MED, so a 250-step post-meal walk is selected as MVI. Node-confidence and edge-confidence values are updated per and [0078]; synthetic glucose points inserted overnight carry a weight of 0.25 per [0079]. Readiness score R=0.68 supports continuation of moderate-intensity interventions.

Illustration. FIG. 9 schematically depicts the data-flow for the example of [0080]: raw inputs (902)→metric computations (904)→friction & readiness outputs (906)→comparison to MED threshold (908)→intervention delivery (910). Reference numerals 912-918 correspond to the node and edge confidence update blocks.

The computations, variable ranges, and examples disclosed in paragraphs [0073] through [0081] are merely illustrative. Alternate scaling factors, confidence priors, decay constants, or sentiment models may be substituted provided they fulfil the categorical behaviour described herein.

All terms defined in paragraphs [0073] through [0081] are intended for use in claims by reference to the specific calculations, definitions, and examples provided in those paragraphs. Where a claim recites Friction, Readiness, Benefit, MED, node confidence, edge confidence, or synthetic weight, such term shall be construed according to the corresponding paragraph of this section unless expressly stated otherwise.

For avoidance of doubt, implementation of the formulas can be performed on a general-purpose processor executing stored instructions, on dedicated signal-processing hardware, or on any combination thereof. No specific programming language or framework is required.

The foregoing description ensures that a person of ordinary skill in the art can calculate each metric without undue experimentation, satisfying the enablement and written-description requirements of 35 U.S.C. § 112 (a).

TABLE 5-B
End-to-End Example of Metric Computation and Intervention Selection

| | Adh. | Sent. | | Delta_Y | Sigma | | | Ready | |
|---|---|---|---|---|---|---|---|---|---|
| User | (A) | (E) | Friction | (mg/dL) | (mg/dL) | Ben/sigma | Ben/Fric | (R) | MVI/MED |
| M, 45, | 0.80 | 0.80 | 0.20 | −8 | 2 | 4.0 | 20.0 | 0.68 | 250-step |
| mod. | | | | | | | | | walk |
| Activity | | | | | | | | | |

### Synthetic Proof-of-Concept Demonstration

In one non-limiting synthetic example, a hypothetical user profile (49-year-old female) generated streaming biometrics over 7 days. A simplified PHKG was constructed with three nodes: resting_heart_rate (Node A), sleep_duration (Node B), and postprandial_glucose (Node C), each initialized with a confidence of 0.5.

### Stage-by-Stage Causal Analysis:

PHKG Ingestion
   A: [70, 72, 75, 73, 74, 76, 78] bpm
   B: [7.0, 6.5, 6.0, 7.2, 7.5, 6.8, 6.9] hrs
   C: [140, 150, 155, 145, 148, 152, 158] mg/dL
Post-PHKG Temporal Inference
   Trends: A_trend (Increase), B_trend (Decrease), C_trend (Increase)
GAM Modeling (Preliminary)
   Fitted dose-response: A to C with slope approximately 1.2 mg/dL per bpm
   B to C pruned (flat relationship)
Mutual Information
   MI(A, C)=0.35 (retained)
   MI(B, C)=0.05 (pruned)
GAM (Refined)
   Confirms A→C direction (p<0.01)
DAG-GNN Learning
   Edge learned: A→C
   Weight: w=0.9, confidence updated to 0.65
Counterfactual Simulation

$$Delta\_A = - 5 bpm -> predicted Delta\_C = - 6 mg / dL ( sigma = 1.2 )$$

Posterior edge confidence updated to 0.75
Recommendation Outcome

$$Benefit = 6 / 1.2 = 5$$

$$Friction = 0.4$$

MED Intervention: “Add 10 minute low intensity walk to reduce resting heart rate and improve postprandial glucose control.

### Synthetic Enablement Example (Proof of Functionality)

| | | | Detected | | | |
|---|---|---|---|---|---|---|
| User | Data | Metric | Causal | Predicted | Delivered | Outcome |
| Profile | Duration | Tracked | Link | Change | MED | Shift |
| Female, 49 | 7 days | HR, Sleep, | Resting HR −> | −6 mg/dL | 10-min | Predicted |
| | | PPG | PPG | | walk | Delta |
| | | | | | | PPG = −6 +/− |
| | | | | | | 1.2 |

### Enablement Demonstration Table

| Functional Block | Input | Output/Result | Evidence Type |
|---|---|---|---|
| PHKG Ingestion | 7-day HRV, Sleep, | Nodes created with | Synthetic [0086] |
| | glucose | initial confidence = 0.5 | |
| Post-PHKG Inference | Value Arrays | A_trend Increase, | Trend engine [0087] |
| | | B_trend Decrease, | |
| | | C_trend Increase | |
| Causal Modeling | A −> C | Dose-response slope ≈ | GAM Fitting [0087] |
| | | 1.2 mg/dL | |
| Mutual Info + | A <− −> C retained, B | MI (A, C) = 0.35, MI | Validation [0087] |
| CAM | <− −> C pruned. | (B, C) = 0.05, CAM | |
| | | confirms A −> C | |
| DAG-GNN + | A −> C (w = 0.9) | Delta_A = −5 bpm −> | Bayesian update shown |
| Counterfactual | | Delta_C = −6 mg/dL | |
| Recommendation | Causal Impact + | MED intervention = 10 | Benefit/Friction = 5 |
| Engine | friction = 0.4 | min walk selected | |
| RL Adaptation | Adherence confirmed | Confidence edge | Reinforcement |
| | | increased to 0.75 | Learning |

### ALTERNATIVE EMBODIMENTS

On-device threshold detection. A mobile embodiment computes deep-sleep percentage locally and triggers screen-off micro-challenges without network connectivity.
Federated inference. PHKG shards reside on-device; only anonymized edge gradients are aggregated in the cloud.
Wellness-only mode. If no clinical or medication data are present, the causal stack operates solely on wearable and conversational inputs, outputting lifestyle-focused recommendations.
Flexible Tailoring Embodiment. In some configurations, the system does not enforce strict phase progression. Instead, it applies lightweight nudges or motivational micro-challenges that adapt to emotional readiness or contextual patterns, without assuming an MED-to-Optimal lifecycle.

The invention is thus not limited to the disclosed embodiments but encompasses all modifications and equivalents falling within the scope of the appended claims.

### Statutory Eligibility Anchor (§ 101 Compliance)

To the extent that any portion of this invention may be characterized as abstract or algorithmic, this disclosure expressly anchors the claimed system to practical, physical components. The claimed invention (i) ingests real-world physiological data via wearable sensors and consumer medical devices; (ii) stores and updates structured causal graphs (PHKGs) in tangible memory; (iii) uses machine-executed simulation and reinforcement-learning policies to identify minimum-dose interventions; and (iv) outputs tailored health actions through tangible user interfaces. Furthermore, as illustrated in FIG. 8 and FIG. 9 and recited in the associated claims, the system performs a concrete sequence of machine-executed steps producing measurable, real-world outcomes. Accordingly, the claimed invention satisfies both steps of the Alice/Mayo test and falls within the statutory categories of machine, manufacture, and process.

Differentiation from Prior Art

Unlike prior systems such as US 2024/0047042 A1 (“Methods and Systems for Generating Personalized Treatments via Causal Inference”), which rely primarily on Model-Twin Randomization (MoTR) after fitting outcome models and therefore lack any layered, sample-gated causal validation, the present invention first constructs an evolving personal health knowledge graph (PHKG) that stores metric values, provenance, and node-level Bayesian confidence labels. This structured substrate enables a multi-layer causal-inference pipeline-Mutual-Information (MI) discovery→GAM modeling→Neural-Granger causality→CAM filtering→DAG-GNN structure learning→deep counterfactual simulation, across heterogeneous, multi-domain health metrics.

In contrast to U.S. Pat. No. 10,881,463 B2 (“Optimizing Patient Treatment Recommendations Using Reinforcement Learning Combined with Recurrent Neural Network Patient State Simulation”), whose RL agent operates on unstructured EHR sequences without dose calibration or causal feedback, the present invention filters and ranks interventions with a friction-weighted benefit score and selects the minimum-effective-dose (MED) that surpasses a safety and benefit threshold. The synergy of PHKG structure, layered causal discovery, and friction-calibrated ranking enables real-time, multi-domain personalization that existing RL-only systems do not provide.

While U.S. Pat. No. 11,586,997 B2 (“Value of Future Adherence”) introduces monetary adherence scoring, it neither validates causality nor computes a benefit-over-friction ratio. References such as U.S. Pat. No. 10,360,349 B2 (“Personalized Medicine Service”) and Gyrard 2018 (“Personalized Health Knowledge Graph”) build domain knowledge graphs, yet they rely on heuristic ranking scores and omit Bayesian node-level confidence updates, staged causal validation, and any friction-based dose optimization.

U.S. Pat. No. 11,860,720 B2 (“Non-linear Causal Modeling from Diverse Data Sources”) teaches a sequential pipeline that combines Neural-Granger modeling with other causal estimators. However, it provides no PHKG structure, applies no sample-gating, and omits any friction- or MED-based intervention ranking.

Recent digital-twin and graph-based optimization efforts likewise distinguish the present invention:

US 2021/0196195 A1 (“Precision Treatment with Machine Learning and Digital Twin Technology for Optimal Metabolic Outcomes”) constructs a multi-modal metabolic digital twin that forecasts glucose and lipid states. It remains purely predictive, lacks a layered causal-discovery stack, stores no node-level Bayesian confidence, and does not enforce a benefit-over-friction dose gate.

WO 2023/239647 A2 (“Systems and Methods to Measure, Predict and Optimize Brain Function”) relies primarily on reinforcement-learning optimization inside a neuro-stimulation twin and lacks layered, sample-gated causal validation.

arXiv 2503.00134 (2025, “Personalized Causal Graph Reasoning for LLMs: A Case Study on Dietary Recommendations”) constructs user-specific causal graphs for coaching, yet does not incorporate MED/friction logic, PHKG schema, or safety gating.

IEEE Access 12:61810 (2024, “CauseKG: A Framework Enhancing Causal Inference With Implicit Knowledge Deduced From Knowledge Graphs”) presents a general framework that integrates KG semantics into causal-inference algorithms, demonstrating superior causal-estimation accuracy on both synthetic and real-world datasets, but remains domain-agnostic, and provides no mechanism for recommending or ranking interventions, nor any adaptive behavioral-feedback loop.

Recent academic efforts further underscore the distinction.
Yang, F. & Rahmani, H. “Personalized Causal Graph Reasoning for LLMs: A Case Study on Dietary Recommendations,” arXiv 2503.00134 v1, 2025, §§ 3-4, FIGS. 1-2. The authors generate a per-user causal graph and run a single-pass PC algorithm plus a counter-factual test to select meals, but they provide no sample-gated causal pipeline, Bayesian node-confidence updates, or MED/friction-based intervention ranking.
Mishra, A. & Shridevi, S. “Knowledge-Graph-Driven Medicine Recommendation System Using GNNs on Longitudinal Medical Records,” Scientific Reports 14:25449, 2024, Abstract, § “KGDNet”, Table 1, FIG. 5a. Their system learns GNN embeddings over visit-level medical knowledge graphs to recommend drugs while controlling drug-drug interactions, yet it lacks any layered causal-validation pipeline or adaptive dose optimisation logic.
Zheng, Z. et al. “CI-GNN: A Granger-Causality-Inspired Graph Neural Network for Interpretable Brain-Network-Based Psychiatric Diagnosis,” arXiv 2301.01642 v3, 2024, Abstract, § 3. CI-GNN identifies causal sub-graphs through conditional-MI regularisation but does not address intervention ranking, MED enforcement, or PHKG-style updates.
McVay, M. et al. “Dose-Response Research in Digital Health Interventions: Concepts, Considerations, and Challenges,” Health Psychology 38 (12): 1168-1174, 2019, pp 1169-1171; and Chen, X. & Schmidt, L. “Exploring a Behavioral Model of ‘Positive Friction’ in Human-AI Interaction,” arXiv 2402.09683 v1, 2024, §§ 2-4. These works discuss dosage tuning and friction as theoretical constructs, but neither integrates them into a real-time, graph-based optimization loop with causal validation.

Accordingly, no known reference discloses the unique combination of
1. an evolving PHKG with node-level causal roles and Bayesian confidence,
2. a sample-gated, multi-layer causal-inference stack,
3. friction- and benefit-weighted intervention ranking that enforces MED,
4. plug-in optimization across multiple domains and conditions, and
5. real-time adaptive learning that preserves clinical safety.
   These features render the present invention non-obvious over both traditional knowledge-graph systems and the latest digital-twin, GNN, or LLM-based approaches.

What is claimed is:

1. A computer-implemented system for personalized health optimization, comprising: (a) a data acquisition layer (100) including a wearable-sensor interface module configured to receive time-stamped physiological, behavioral, or pharmacological data from one or more wearable sensors and external data sources; (b) a non-transitory memory storing a personalized health knowledge graph (PHKG) (120) for each user, the PHKG comprising nodes comprising: (i) nodes representing observed health metrics labeled with causal role selected from a configurable set comprising at least driver, outcome, moderator and confounder, and (ii) directed edges weighted by estimated causal strength and annotated with edge confidence scores derived from multi-stage inference; (c) one or more processors configured to: (i) execute a sample-gated, multi-stage causal-inference stack (140) on the PHKG, the stack comprising: (A) a first stage that applies generalized additive modeling (GAM) when a first sample-count threshold stored in non-transitory memory is satisfied; (B) a second stage that applies mutual-information analysis, Neural Granger causality, and contrastive attribution methods when both a second sample-count threshold and a sampling-uniformity threshold are satisfied; and (C) a third stage that applies directed-acyclic-graph neural-network (DAG-GNN) learning and counterfactual simulation when a third sample-count threshold and a sampling-uniformity threshold are satisfied; wherein said thresholds are modifiable parameters and the stack identifies at least one modifiable driver of a target outcome; and (ii) generate a behavioral readiness score based on adherence history, sentiment, and intent; and (iii) calculate a friction score based on at least one of adherence history, predicted effort, or user-reported burden; and (d) a recommendation engine (150) configured to rank a plurality of candidate interventions by a benefit-to-friction ratio, enforce clinical safety constraints, and select the top-ranked intervention, the intervention being selected from a group comprising: (i) Minimum Effective Dose (MED); (ii) adaptive low-dose; (iii) optimal-effort action; and (iv) a behaviorally tailored challenge; and (v) a continuous, phase-less, or micro-challenge intervention; wherein said engine suppresses all candidate interventions whose benefit-to-friction falls below a defined threshold or violates safety constraints.

2. A method of generating an individualized health intervention comprising: (a) acquiring heterogeneous, time-stamped data from one or more sensors, medical devices, laboratory systems, or conversational inputs; (b) constructing a confidence-weighted PHKG in accordance with claim 1; (c) executing the sample-gated multi-stage causal-inference stack to determine at least one modifiable driver of a selected target outcome; and (d) selecting and delivering a minimum viable intervention, wherein the minimum viable intervention corresponds to a Minimum Effective Dose (MED) whose benefit-to-friction ratio exceeds a predefined threshold.

3. A non-transitory computer-readable medium storing instructions that, when executed by one or more processors, cause a system to: (a) receive heterogeneous, time-stamped data from sensors, medical devices, laboratory systems, or conversational inputs; (b) construct a confidence-weighted personal health knowledge graph (PHKG); (c) execute the sample-gated multi-stage causal-inference stack to identify at least one modifiable driver of a selected target metric; (d) select and deliver a safety-constrained, minimum-viable intervention based on (i) a benefit-to-friction ranking, (ii) friction score, and (iii) user readiness.

4. A computer-implemented system comprising a personal health knowledge graph (PHKG) engine configured to: (a) construct, for each user, a directed graph in which each node stores a metric value, a timestamp, a source type, and a causal-role label; (b) prevent insertion of duplicate nodes by hashing a tuple consisting of the metric type and a timestamp granularity; and (c) store, for each edge of the directed graph, a causal weight and a confidence value that is updated by Bayesian evidence accumulation based on observed outcomes.

5. The system of claim 1, wherein the PHKG comprises nodes, each of which stores: (a) a time-stamped value history; (b) a source-type label selected from the group consisting of direct, inferred, and synthetic; and (c) a causal-role label selected from the group consisting of driver, amplifier, mediator, latent, and outcome.

6. The system of claim 5, wherein each node is uniquely identified based on a hashed tuple comprising the metric type and timestamp granularity.

7. The system of claim 5, wherein edges between nodes store a causal weight (w) and a confidence value (C), and said confidence is updated using Bayesian accumulation based on evidence sources selected from: (a) mutual-information analysis (MI); (b) generalized additive modeling (GAM); (c) Neural Granger causality; (d) contrastive attribution methods (CAM); (e) directed-acyclic-graph neural-network learning (DAG-GNN); and (f) Bayesian counterfactual simulation applied to PHKG.

8. The system of claim 1, wherein execution of the multi-stage causal-inference stack is gated by stage-specific sample-count and sampling-uniformity criteria maintained in non-transitory memory, such that each stage only executes when its corresponding criteria are satisfied.

9. The system of claim 8, wherein the “first stage” is executed by applying a generalized additive model (GAM) to the PHKG upon each data update when at least two (2) metric samples are present.

10. The system of claim 8, wherein: (a) the first stage's sample-count criterion defaults to at least two valid samples; (b) the second stage's sample-count criterion defaults to at least seven valid samples within a rolling seven-day window and its sampling-uniformity criterion defaults to a coefficient of variation not exceeding twenty-five percent; and (c) the third stage's sample-count criterion defaults to at least thirty valid samples within a rolling fourteen-day window and its sampling-uniformity criterion defaults to a coefficient of variation not exceeding twenty-five percent.

11. The system of claim 8, wherein the “second stage” comprises (i) mutual-information analysis, (ii) generalized additive modeling (GAM), (iii) Neural Granger causality, and (iv) contrastive attribution methods (CAM), each executed when both (A) a sample-count threshold is satisfied, defaulting to at least seven (7) samples collected within a rolling seven-day window; and (B) a sampling-uniformity threshold is satisfied, defaulting to a coefficient of variation of the sampling intervals not exceeding twenty-five percent (25%), wherein these thresholds are stored as modifiable parameters in non-transitory memory.

12. The system of claim 8, wherein the “third stage” comprises (a) directed-acyclic-graph neural-network learning (DAG-GNN) and (b) deep counterfactual simulation that tests end-to-end intervention efficacy, each executed only when both (i) a third sample-count threshold is met, defaulting to at least thirty (30) samples collected within a rolling fourteen-day window, and (ii) a third sampling-uniformity threshold is met, defaulting to a coefficient of variation of sampling intervals not exceeding twenty-five percent (25%), wherein these thresholds are stored as modifiable parameters in non-transitory memory.

13. The system of claim 1, wherein user-specific friction is computed from adherence history, engagement signals, and sentiment analysis.

14. The method of claim 2, further comprising suppressing any intervention whose simulated impact violates a personalized safety limit.

15. The system of claim 1, wherein the set V of nodes in the personalized health knowledge graph comprises at least one node representing a medication dose, a medication schedule, or a pharmacological class.

16. The method of claim 2, further comprising simulating alternate medication dosages or administration times to identify a personalized titration plan.

17. The system of claim 1, wherein the PHKG simultaneously represents nodes associated with at least two distinct health domains selected from: metabolic, cardiovascular, sleep, stress, renal, dermatological.

18. The system of claim 1, wherein the multi-stage causal-inference stack identifies chained causal relationships traversing two or more distinct health domains and employs a domain-agnostic utility function that synthesizes causal influence metrics across the chained relationships to optimize cross-domain interventions.

19. The system of claim 1, wherein the system optionally comprises a reinforcement-learning module that adjusts intervention timing or intensity based on a reward signal derived from verified outcome change and adherence.

20. The system of claim 1, wherein PHKG updates and causal-inference processing are performed either via federated learning using anonymized gradients or in an offline-batch mode when connectivity is unavailable.

21. The method of claim 2, wherein synthetic observations are down-weighted relative to real observations using a weighting factor inversely proportional to real-observation count.

22. The system of claim 1, wherein: (a) each target metric, including, but not limited to, biomarkers, behavioral indicators, or other domain-specific outcomes, is dynamically declared at runtime through a modular plug-in schema, thereby enabling the system to optimize for any health condition or intervention goal for which compatible input and outcome data are available; and (b) a conversational interface is configured to (i) explain recommended interventions using natural-language generation, (ii) extract sentiment, intent, or adherence feedback from user dialogue, and (iii) update personalization logic or PHKG parameters based on the extracted feedback.

23. The system of claim 18, wherein the reinforcement-learning module adjusts intervention intensity, challenge framing, or fallback selection based on detected behavioral patterns, emotional sentiment, or motivational state derived from engagement history, free-text input, or conversational analysis.

24. The system of claim 1, further comprising a fallback module configured to apply rule-based heuristics or simplified statistical models in the event that target metrics or driver candidates fail to meet minimum data thresholds for causal inference.
